(19) United States
(12) Patent Application Publication
(10) Pub. No.: US 2003/0009120 A1
(43) Pub. Date: Jan. 9, 2003

(54) Device for prevention of abnormal joint rotation

(76) Inventor: Doris MacAllister, North Quincy, MA (US)

Correspondence Address: LAMBERT & ASSOCIATES, P.L.L.C., 92 STATE STREET, BOSTON, MA, 02109-2004, US

(21) Appl. No.: 10/065,248

(22) Filed: Sep. 27, 2002

Related U.S. Application Data
(63) Continuation-in-part of application No. 09/735,965, filed on Dec. 13, 2000.

(51) Int. Cl. A61F 5/00; A61F 13/00

(52) U.S. Cl. 602/23; 602/61

(57) ABSTRACT

The device for prevention of the abnormal joint rotation is comprised of an appendage securing section and a torso securing section. The appendage securing section is constructed and arranged to secure to the appendage having the abnormal joint rotation. The torso securing section is constructed and arranged to secure to the torso of the patient and to apply correcting pressure directed upon the appendage securing section, wherein the correcting pressure exerted by the torso securing section upon the appendage securing section counters the direction of the abnormal joint rotation. The torso securing section has means for coupling to the appendage securing section. In a single unit embodiment, the appendage securing section and the torso securing section are joined to form a wrap. Different methods of application of the wrap to patient's body could be used for prevention of either the external or internal rotation of the hip by providing directed pressure upon a leg suffering from the abnormal hip rotation.

### CROSS REFERENCE TO RELATED APPLICATIONS

This application claims the benefit in the form of a continuation-in-part under 37 C.F.R. 1.53(b)(1) of currently pending parent application Ser. No. 09/735,965 filed Dec. 13, 2000, by the same inventor.

### BACKGROUND OF INVENTION

The present invention relates to a device and methods aimed at providing a correcting pressure upon an appendage to counter abnormal joint rotation to promote proper joint alignment and reduce pain.

Hip alignment shifts can result from injury, loss of bone density, arthritis or the aging process. One such change in hip alignment is hip rotation. People with hip rotation suffer from pain and loss of mobility stemming from a change in their hip alignment causing a tendency for the femur to rotate inward or outward. Additionally, patients may overcompensate with their back and knees in order to place less pressure on their hips, thus causing further pain. Numerous individuals suffer from hip pain caused by hip rotation and believe that their only options are either medication or surgery.

Prior art hip stabilizing devices provide for the rehabilitation of soft tissue injuries such as muscle injuries but do not aim at prevention of abnormal joint rotation.

The following prior art is known to Applicant: U.S. Pat. No, 5,188,585 to Peters discloses lumbo-sacral support device formed by the combination of a lumbo-sacral belt adopted to encircle the torso of the patient and a separate compression belt, which device is placed over the lower torso region of the patient to alleviate pain resulting from injury, for preventing injury, to improve posture, and to support sagging or weak muscles. The device disclosed by Peters does not aim at prevention of abnormal joint rotation and it's design does not allow it to be used for prevention of abnormal joint rotation. The pressure exerted by the Peter's device is internally directed and cannot have much effect on the direction of hip rotation. On the other hand, the present invention provides for a directional application of pressure specifically aimed at countering direction of the abnormal joint rotation.

U.S. Pat. No. 5,267,928 to Barile, discloses a support garment composed of short pants with various straps to assist in the healing of soft tissue. Barile's device is aimed at providing internally directed pressure upon thighs to promote healing of soft tissue. However, Barile does not disclose an application of his device for prevention of the abnormal joint rotation and the design of his device prevents it from functioning to counter direction of the abnormal joint rotation. Although Barile discloses wrapping of a strap around the thigh and the lower torso, his wrapping method is not aimed at and does not induce directed pressure upon a joint having abnormal rotation. Instead, it only aims at providing inwardly directed pressure upon soft tissues to aid in their healing. In comparison, the presently disclosed device provides a directed pressure upon an appendage, which pressure serves to counter the direction of the abnormal joint rotation, a feature that is not disclosed, foreseen, or enabled by Barile.

U.S. Pat. No. 3,529,601 to Kirkland, discloses a bandage that can be wrapped around parts of human body to induce perspiration for weight loss. Kirkland's device is not aimed at prevention of abnormal joint rotation and his device and wrapping methods do not inherently function in prevention of abnormal joint rotation.

U.S. Pat. No. 5,993,375, to Engel, discloses a pad having a plurality of magnets within the pad. Engel's pad is aimed at providing a tool for magnetic therapy. Although, the presently disclosed invention could be used with magnetic bodies in order to provide added therapeutic properties, such use of magnets is not central to the concept of the present invention, which is to provide a device that would counter the direction of the abnormal rotation of a joint.

U.S. Pat. No. 5,423,852, to Daneshvar, discloses a wrap that is positioned around thigh and abdomen. Daneshvar's wrap is aimed at giving support to a balloon used in prevention of bleeding from a wound created a consequence of catheterization of a vessel proximate to the groin. The design of Daneshvar's wrap does not function in prevention of abnormal joint rotation, nor is such application envisioned in Daneshvar.

The present invention substantially differs from the teachings of the above referenced prior art in that it is designed for and aimed at correction of joint alignment by providing a correcting counter pressure upon an appendage suffering from the abnormal joint rotation. The presently disclosed device can be adjusted to any size patient, can be easily concealed under clothing, and does not interfere with a normal range of motion.

### SUMMARY OF INVENTION

The disclosed device and methods are aimed at providing a correcting pressure upon an appendage to counter abnormal joint rotation.

The device for prevention of the abnormal joint rotation is comprised of an appendage securing section and a torso securing section. The appendage securing section is constructed and arranged to secure to the appendage having the abnormal joint rotation. The appendage having an abnormal joint rotation could be a leg or an arm. The appendage securing section is secured to the appendage by placement of the appendage securing section around the appendage at a position proximal to a position of the joint having abnormal rotation.

The torso securing section is constructed and arranged to secure to the torso of the patient and to apply correcting pressure directed upon the appendage securing section, wherein the correcting pressure exerted by the torso securing section upon the appendage securing section counters the direction of the abnormal joint rotation.

The torso securing section has any means well known in the art, such as hook and loop means, for coupling to the appendage securing section. The torso securing section has a securing end, wherein the securing end of the torso securing section has any means well known in the art, such as hook and loop means, for coupling to the appendage securing section.

The appendage securing section could have a first end and a second end, while the torso securing section could have a first end, an intermediate section, and a securing end, wherein the securing end has means well known in the art, such as hook and loop means, for coupling to the appendage securing section. In a single unit embodiment, the second end of the appendage securing section is joined to the first end of the torso securing section thereby forming a wrap.

The wrap could be used in disclosed methods for prevention of either the external or internal rotation of the hip by providing directed pressure upon a leg suffering from the abnormal hip rotation.

Therefore, it is the object of the present invention to provide a device for prevention of abnormal joint rotation.

It is another object of the present invention to provide a device for prevention of external rotation of the hip.

It is another object of the present invention to provide a device for prevention of internal rotation of the hip.

It is another object of the present invention is to provide a method of use of the disclosed device for prevention of external rotation of the hip.

Yet another object of the present invention is to provide a method of use of the disclosed device for prevention of internal rotation of the hip.

These and other features, aspects and advantages of the present invention will become better understood with reference to the following description, appended claims, and accompanying drawings.

### BRIEF DESCRIPTION OF DRAWINGS

FIG. 1 depicts a front view of one of the embodiments of the invention aimed at prevention of the external rotation of the hip;

FIG. 2 depicts a front view of one of the embodiments of the invention aimed at prevention of the internal rotation of the hip;

FIG. 3 depicts a front view of a wrap, a single unit embodiment of the invention, positioned on a patient for prevention of the external rotation of the hip;

FIG. 4 depicts a front view of a wrap, a single unit embodiment of the invention, positioned on a patient for prevention of the internal rotation of the hip;

FIG. 5 depicts a front view of the wrap;

FIGS. 6-9 depict steps in a method of use of the wrap on a patient for prevention of the external rotation of the hip;

FIGS. 10-14 depict steps in a method of use of the wrap on a patient for prevention of the internal rotation of the hip.

### DETAILED DESCRIPTION

FIGS. 1-5 depict embodiments of the device for prevention of the abnormal joint rotation, while FIGS. 6-14 depict steps in the methods of use of the disclosed device.

The device for prevention of the abnormal joint rotation is comprised of an appendage securing section 1 and a torso securing section 2. The appendage securing section 1 is constructed and arranged to secure to the appendage having the abnormal joint rotation. The appendage having an abnormal joint rotation could be a leg or an arm. FIGS. 1 and 2 depict the appendage securing section 1 secured to the appendage by placement of the appendage securing section 7 around the appendage at a position proximal to a position of the joint having abnormal rotation. Referring to FIGS. 1 and 2, the joint having an abnormal joint rotation is a hip and the appendage around which the appendage securing section 1 is placed is a leg. Specifically, the appendage securing section 1 could be placed around a thigh of the leg, as shown in FIGS. 1 and 2.

The torso securing section 2 is constructed and arranged to secure to the torso of the patient and to apply correcting pressure directed upon the appendage securing section 1, wherein the correcting pressure exerted by the torso securing section 2 upon the appendage securing section 1 counters the direction of the abnormal joint rotation.

The torso securing section 2 has any means well known in the art, such as hook and loop means, for coupling to the appendage securing section 1. FIGS. 1 and 2 depict the torso securing section 2 secured to patient's torso by placement of the torso securing section 2 around the torso of the patient. The torso securing section 2 has a securing end 10, wherein the securing end 10 of the torso securing section 1 has any means well known in the art, such as hook and loop means, for coupling to the appendage securing section 1.

FIG. 1 depicts an embodiment of the invention aimed at prevention of an external rotation of the hip. The direction of the external rotation of the hip is shown by arrow 20, where the hip joint has a propensity to rotate outwardly (clockwise) with respect to the vertical axis 50 of the hip. Here, the torso securing section 2 exerts pressure upon the appendage securing section 1, which in turn exerts a correcting pressure upon the thigh of the leg. The direction of the correcting pressure is shown by arrow 30.

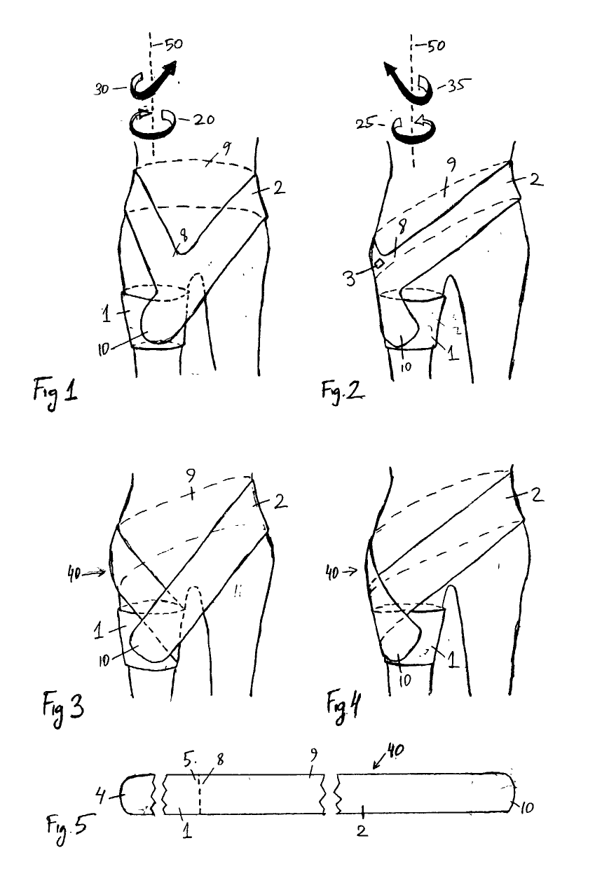

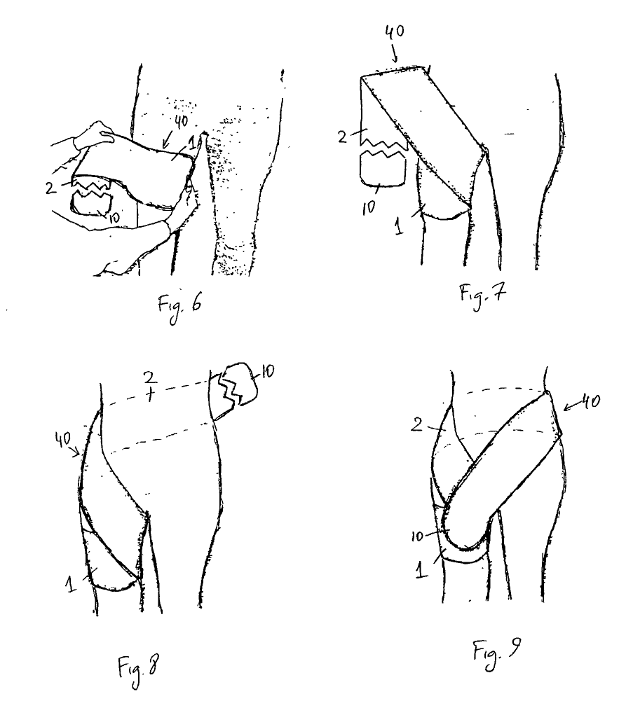

The correcting pressure upon the thigh could be inwardly directed in relation to the hip having external rotation with a result of prevention of the external rotation of the hip. Alternatively, the correcting pressure could be simultaneously inwardly, frontally and upwardly directed in relation to the hip having external rotation, as achieved by the embodiments shown in FIGS. 1 and 3, where direction of the correcting pressure is shown by arrow 30 in FIG. 7.

Similarly, FIG. 2 depicts an embodiment of the invention aimed at prevention of an internal rotation of the hip. The direction of the internal rotation of the hip is shown by arrow 25, where the hip joint has a propensity to rotate inwardly (counterclockwise) with respect to the vertical axis 50 of the hip. The torso securing section 2 exerts pressure upon the appendage securing section 1, which in turn exerts a correcting pressure upon the thigh of the leg. The direction of the correcting pressure is shown by arrow 35.

The correcting pressure upon the thigh could be outwardly directed in relation to the hip having internal rotation with a result of prevention of the internal rotation of the hip. Alternatively, the correcting pressure could be simultaneously outwardly, rearwardly and upwardly directed in relation to the hip having internal rotation, as achieved by the embodiments shown in FIGS. 2 and 4, where direction of the correcting pressure is shown by arrow 35 in FIG. 2.

It is preferred that the torso securing section 2 be composed of an elastic material. Elasticity of the torso securing section 2 aids in exertion of correcting pressure upon the appendage having the abnormal joint rotation.

Additionally, the torso securing section 2 could have means well known in the art, such as a pocket, for affixing upon it of at least one magnetic body 3. Magnetic bodies 3 are frequently used for pain reduction by placing them over body parts suffering from pain.

As previously noted, the disclosed device could be used on almost any appendage suffering from the abnormal joint rotation. Thus, the disclosed device could also be applied to an arm, wherein the joint having the abnormal rotation is a shoulder.

As shown in FIGS. 1-5, the appendage securing section 1 could have a first end 4 and a second end 5, and the torso securing section 2 could have a first end 8, an intermediate section 9, and a securing end 10, wherein the securing end 10 has means well known in the art, such as hook and loop means, for coupling to the appendage securing section 1.

The appendage securing section 1 could have means well known in the art, such as hook and loop means, for coupling of the first end 4 of the appendage securing section to the second end 5 of the appendage securing section 7. Alternatively, the first end 4 of the appendage securing section 1 could be coupled to the second end 5 of the appendage securing section 1 as shown in FIGS. 1 and 2.

The torso securing section 2 could have means well known in the art, such as hook and loop means, for coupling of the first end 8 of the torso securing section 2 to the intermediate section 9 of the torso securing section 1. Alternatively, the first end 8 of the torso securing section 7 could be coupled to the intermediate section 9 of the torso securing section 1 as depicted in FIGS. 1 and 2.

In a single unit embodiment, the second end 5 of the appendage securing section 1 is joined to the first end 8 of the torso securing section 1 thereby forming a wrap 40 depicted in FIGS. 3-14.

The wrap 40 could be used in a method of prevention of the external rotation of the hip by providing directed pressure upon a leg suffering from the external rotation of the hip. The method comprises the following steps shown in FIGS. 6-9:

a) placing the first end 4 of the appendage securing section 1 of the wrap 40 against an inside of patient's thigh of the leg suffering from the external rotation of the hip, wherein the appendage securing section 1 of the wrap 40 is positioned in front of the thigh, as shown in FIG. 6;

b) wrapping the appendage securing section 1 of the wrap 40 at least once around the thigh, as shown in FIG. 7;

c) wrapping the torso securing section 2 at least once around patient's torso by bringing the torso securing section 2 of the wrap 40 up and over the lateral side of the hip suffering from the external rotation of the hip, over patient's lower back, and over patient's lateral side which lateral side is opposed to the side suffering from the external rotation of the hip, as shown in FIGS. 8 and 9; and

d) coupling of the securing end 0 of the torso securing section 2 of the wrap 40 to the appendage securing section 1 at the front of the thigh, as shown in FIG. 9.

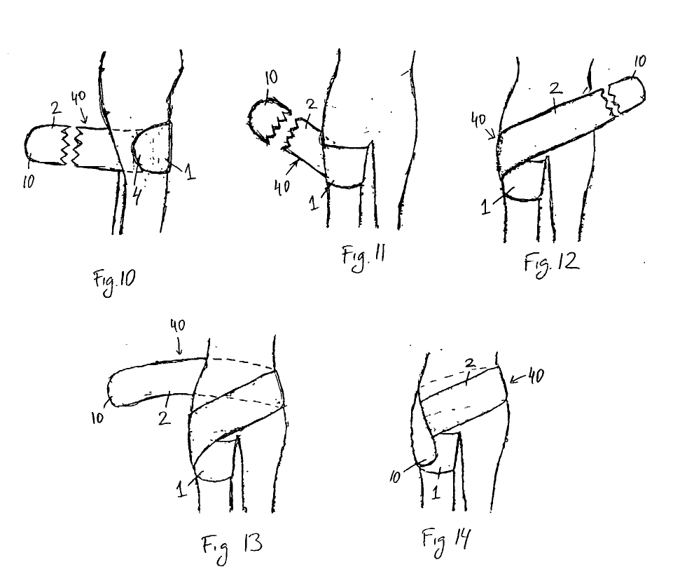

The wrap 40 could also be used in a method of prevention of the internal rotation of the hip by providing directed pressure upon a leg suffering from the internal rotation of the hip. The method comprises the following steps shown in FIGS. 10-14:

a) placing the first end 4 of the appendage securing section 1 of the wrap 40 against an outside of patient's thigh of the leg suffering from the external rotation of the hip, wherein the appendage securing section 1 of the wrap 40 is positioned in front of the thigh, as shown in FIG. 10;

b) wrapping the appendage securing section 1 of the wrap 40 at least once around the thigh, as shown in FIGS. 10-12;

c) wrapping the torso securing section 2 at least once around patient's torso by bringing the torso securing section 2 of the wrap 40 up and over the front of the thigh and over the abdomen, over patient's lateral side which lateral side is opposed to the side suffering from the internal rotation of the hip, and over patient's lower back, as shown in FIGS. 12-14; and

d) coupling of the securing end 10 of the torso securing section 2 of the wrap 40 to the appendage securing section 1 at the front of the thigh, as shown in FIG. 14.

After the wrap 40 is properly positioned on the body of the patient, as shown in FIGS. 3 and 4, the wrap 40 provides same therapeutic characteristics as those of the embodiments described above and depicted in FIGS. 1 and 2. FIG. 3 depicts the wrap 40 applied for treatment of the external rotation of the hip, while FIG. 4 depicts the wrap 40 applied for treatment of the internal rotation of the hip.

It will be further understood that the embodiments described herein are merely exemplary and that a person skilled in the art may make variations and modifications without departing from the spirit and scope of the invention. All such variations and modifications are intended to be included within the scope of the appended claims.

What is claimed is:

1. A device for prevention of the abnormal joint rotation, comprising:
an appendage securing section constructed and arranged to secure to the appendage having the abnormal joint rotation;
a torso securing section having means for coupling to the appendage securing section, wherein the torso securing section is constructed and arranged to secure to the torso of the patient and to apply correcting pressure directed upon the appendage securing section, wherein the correcting pressure exerted by the torso securing section upon the appendage securing section counters the direction of the abnormal joint rotation.

2. The device of claim 1 wherein:
the torso securing section is constructed and arranged to secure to the torso of the patient and to apply correcting pressure upon the appendage securing section, wherein the correcting pressure exerted by the torso securing section upon the appendage securing section counters the direction of the external rotation of the joint.

3. The device of claim 1 wherein:
the torso securing section is constructed and arranged to secure to the torso of the patient and to apply pressure upon the appendage securing section, wherein the correcting pressure exerted by the torso securing section upon the appendage securing section counters the direction of the internal rotation of the joint.

4. The device of claim 1 wherein:
the torso securing section has means for affixing upon it of at least one magnetic body.

5. The device of claim 1 wherein:
the appendage suffering from the abnormal joint rotation is a leg, wherein the joint having the abnormal rotation is a hip.

6. The device of claim 1 wherein:
the appendage suffering from the abnormal joint rotation is an arm, wherein the joint having the abnormal rotation is a shoulder.

7. The device of claim 1 wherein:
the torso securing section is composed of an elastic material.

8. The device of claim 1 wherein:
an appendage securing section constructed and arranged to secure to a thigh of a leg having the abnormal rotation of the hip; and
a torso securing section is constructed and arranged to secure to the torso of the patient and to apply correcting pressure directed upon the appendage securing section, wherein the correcting pressure exerted by the torso securing section upon the appendage securing section counters the direction of the abnormal rotation of the hip.

9. The device of claim 8 wherein:
the torso securing section is constructed and arranged to secure to the torso of the patient and to apply pressure upon the appendage securing section, wherein such pressure is inwardly directed in relation to the hip having external rotation with a result of prevention of the external rotation of the hip.

10. The device of claim 8 wherein:
the torso securing section is constructed and arranged to secure to the torso of the patient and to apply pressure upon the appendage securing section, wherein such pressure is simultaneously inwardly, frontally and upwardly directed in relation to the hip having external rotation with a result of prevention of the external rotation of the hip.

11. The device of claim 8 wherein:
the torso securing section is constructed and arranged to secure to the torso of the patient and to apply pressure upon the appendage securing section, wherein such pressure is outwardly directed in relation to the hip having internal rotation with a result of prevention of the internal rotation of the hip.

12. The device of claim 8 wherein:
the torso securing section is constructed and arranged to secure to the torso of the patient and to apply pressure upon the appendage securing section, wherein such pressure is simultaneously outwardly, rearwardly and upwardly directed in relation to the hip having internal rotation, with a result of prevention of the internal rotation of the hip.

13. The device of claim 1 wherein:
the appendage securing section has a first end and a second end; and
the torso securing section has a first end, an intermediate section, and a securing end, wherein the securing end of the torso securing section has means for coupling to the appendage securing section.

14. The device of claim 13 wherein:
the appendage securing section has means for coupling of the first end of the appendage securing section to the second end of the appendage securing section.

15. The device of claim 13 wherein:
the first end of the appendage securing section is coupled to the second end of the appendage securing section.

16. The device of claim 13 wherein:
the torso securing section has means for coupling of the first end of the torso securing section to the intermediate section of the torso securing section.

17. The device of claim 13 wherein:
the first end of the torso securing section is coupled to the intermediate section of the torso securing section.

18. The device of claim 13 wherein:
the second end of the appendage securing section is joined to the first end of the torso securing section thereby forming a wrap.

19. A method of prevention of the external rotation of the hip by providing directed pressure upon a leg suffering from the external rotation of the hip, using a wrap comprising:
the appendage securing section having a first end and a second end;
the torso securing section having a first end, an intermediate section, and a securing end, wherein the securing end of the torso securing section has means for coupling to the appendage securing section; and
wherein the second end of the appendage securing section is joined to the first end of the torso securing section;
said method comprising:
a) placing the first end of the appendage securing section of the wrap against an inside of patient's thigh of the leg suffering from the external rotation of the hip, wherein the appendage securing section of the wrap is positioned in front of the thigh;
b) wrapping the appendage securing section of the wrap at least once around the thigh;
c) wrapping the torso securing section at least once around patient's torso by bringing the torso securing section of the wrap up and over the lateral side of the hip suffering from the external rotation of the hip, over patient's lower back, and over patient's lateral side which lateral side is opposed to the side suffering from the external rotation of the hip; and
d) coupling of the securing end of the torso securing section of the wrap to the appendage securing section at the front of the thigh.

20. A-method of prevention of the internal rotation of the hip by providing directed pressure upon a leg suffering from the internal rotation of the hip, using a wrap comprising:
the appendage securing section having a first end and a second end;
the torso securing section having a first end, an intermediate section, and a securing end, wherein the securing end of the torso securing section has means for coupling to the appendage securing section; and
wherein the second end of the appendage securing section is joined to the first end of the torso securing section;
said method comprising:
a) placing the first end of the appendage securing section of the wrap against an outside of patient's thigh of the leg suffering from the external rotation of the hip, wherein the appendage securing section of the wrap is positioned in front of the thigh;
b) wrapping the appendage securing section of the wrap at least once around the thigh;
c) wrapping the torso securing section at least once around patient's torso by bringing the torso securing section of the wrap up and over the front of the thigh and over the abdomen, over patient's lateral side which lateral side is opposed to the side suffering from the internal rotation of the hip, and over patient's lower back; and
d) coupling of the securing end of the torso securing section of the wrap to the appendage securing section at the front of the thigh.
